(19) United States
(12) Patent Application Publication
(10) Pub. No.: US 2005/0069573 A1
(43) Pub. Date: Mar. 31, 2005

(54) Responsive polymeric system

(75) Inventors: Daniel Cohn, Jerusalem (IL); Alejandro Sosnik, Jerusalem (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(73) Assignee: YISSUM RESEARCH DEVELOPMENT COMPANY OF THE HEBREW UNIVERSITY OF JERUSALEM, JERUSALEM (IL)

(21) Appl. No.: 10/845,476

(22) Filed: May 12, 2004

(30) Foreign Application Priority Data
May 12, 2003 (IL) 155866

(51) Int. Cl. A61F 2/00

(52) U.S. Cl. 424/426

(57) ABSTRACT

A novel environmentally responsive polymeric system is provided for biomedical applications, comprising silicon-containing reactive groups which undergo a hydrolysis-condensation reaction at a predetermined body site and thereby change rheological and mechanical properties of the polymeric system. The polymeric system is useful, for example, as a sealant, as a matrix for drug delivery, in the prevention of post-surgical adhesions, and in gene therapy.

### BACKGROUND OF THE INVENTION

1. Field of the Invention

The present invention relates to a novel organic-inorganic environmentally responsive polymeric system. More specifically, the present invention relates to a responsive polymeric system comprising one or more silicon-containing reactive groups which undergo a hydrolysis-condensation reaction effected primarily at a predetermined body site that results in an increase in the molecular weight due to the polymerization and/or crosslinking of said polymeric system and produces a change in its rheological and mechanical properties, said polymeric system being deployable via a non-invasive or a minimally invasive surgical procedure and useful in a variety of applications, most importantly in the Biomedical field, such as a sealant, as a matrix for drug delivery, in the prevention of post-surgical adhesions and in the Tissue Engineering and Gene Therapy fields.

1. Prior Art

All publications mentioned throughout this application are fully incorporated herein by reference, including all references cited therein.

There is a wide variety of materials which are foreign to the human body and which are used in direct contact with its organs, tissues and fluids. Such materials are called Biomaterials, and they include, among others, polymers, ceramics, biological materials, carbons, metals, composite materials, and combinations thereof.

The development of polymers suitable to be implanted without requiring a surgical procedure, usually named injectable polymers, has triggered much attention in recent years. These materials combine low viscosity at the injection stage, with a gel or solid consistency developed in situ, later on. The systems of the present invention are preferably used, without limitation, as matrices for the controlled release of biologically active agents, as sealants, as coatings and as barriers in the body. The area of Tissue Engineering represents an additional important field of application of the reinforced responsive systems disclosed hereby, where they can perform as the matrix for cell growth and tissue scaffolding.

The syringeability of injectable biomedical systems is their most essential advantage, since it allows their introduction into the body using minimally invasive techniques. Furthermore, their low viscosity and substantial flowability at the administration time, enable them to reach and fill spaces, otherwise unaccessible, as well as to achieve enhanced attachment and improved conformability to the tissues at the implantation site. On the other hand, the sharp increase in rheological and mechanical properties is a fundamental requirement for these materials to be able to fulfil any physical or mechanical function, such as sealing or performing as a barrier between tissue surfaces. The high viscosities attained play also a critical role in generating syringeable materials that, once present at the implantation site, are also able to control the rate of release of drugs or can function as the matrix for cell growth and tissue scaffolding.

Biodegradability plays a unique role in a diversity of devices, implants and prostheses, this property being an additional important requirement for some of these materials. Their most obvious advantage pertains to the fact that there is no need to remove the system, once it has accomplished its objectives. In addition, they can perform as matrices for the release of bioactive molecules and result in improved healing and tissue regeneration processes. Biodegradable polymers such as polyesters of α-hydroxy acids, like lactic acid or glycolic acid, are used in diverse applications such as bioabsorbable surgical sutures and staples, some orthopedic and dental devices, drug delivery systems and more advanced applications such as the absorbable component of selectively biodegradable vascular grafts, or as the temporary scaffold for tissue engineering. The synthesis and biodegradability of poly(lactic acid) was reported by several groups (Kulkarni R. K. and co-workers, Technical Rep. 6608, Walter Reed Army Medical Center, Washington, D.C. (1966); Conn Jr. J. et al, Am. J. Surgery, 128, 19 (1974); Tormala P. and group, Biomaterials, 16, 1353 (1995); Gopferich A., Biomaterials, 17, 103 (1996); Li S., J. Biomed. Mater. Res. (Appl. Biomater.), 48, 342 (1999)). Biodegradable polyanhydrides (Domb A. J. et al, Biomaterials, 11, 690 (1990) and Langer, R., J. Biomed. Mat. Res., 28, 1465 (1994)) and polyorthoesters (Heller J., Biomaterials, 11, 659 (1990) and Gurny R., ‘Polymer Biomaterials in Solution, as Interfaces and as Solids’, Page 683, S. L. Cooper, C. H. Bamford and T. Tsuruta (Editors), VSP-Utrecht, The Netherlands (1995)) having labile backbone linkages, have been developed, the disclosures of which are incorporated herein. Polymers which degrade into naturally occurring materials, such as polyaminoacids, also have been synthesized. Degradable polymers formed by copolymerization of lactide, glycolide, and ε-caprolactone have been disclosed (Kissel T. and collaborators, J. Biomed. Mater. Res., 30, 31-40 (1996)). Polyether-polyester combinations especially of polyethylene glycol (PEG) and aliphatic polyesters like poly(lactic acid), poly(glycolic acid) and poly(caprolactone), either as a blend or as a copolymer, in order to increase the hydrophilicity and degradation rate, have been reported. Most of the work was focused on poly(ethylene glycol)/poly(glycolic) (PEG-PGA) or poly(lactic) (PEG-PLA) acid materials (Cohn et al., Polymer, 28, 2018-2022 (1987) and J. Biomed. Mater. Res., 21, 1301-1316 (1987); Penco et al, J. Appl. Polym. Sci., 78, 1721 (2000); Li S., J. Biomed. Mater. Res. (Appl. Biomater.), 48, 342 (1999); Ronnenberger B. and collaborators, J. Biomed. Mater. Res., 30, 31 (1996); Sawhney S. A. and Hubbell J. A., J. Biomed. Mater. Res. 24, 1397 (1990); Zhu K J and co-workers, J. Appl. Polym. Sci., 39, 1 (1990)). Furthermore, these polymers present relatively fast degradation rates, from a few days to a few months (von Burkersroda F. et al, Biomaterials, 18, 1599 (1997); Penco M. and group, Biomaterials, 17, 1583 (1996)). This drawback constitutes one of the relevant application limitations. Another group of poly(ether-ester)s is the poly(ethylene glycol)-poly(caprolactone) (PEG-PCL)-based polymers. Thus, a broad work was done on high MW PEG-PCL block copolymers. Vert and co-workers (Polym. Int., 45, 419 (1998)) synthesized and characterized PEG-PCL copolymers of intermediate molar masses with both PEG and PCL crystallizable blocks, using dicyclohexylcarbodiimide as coupling agent. Cerrai et al. (J. Mater. Sci.: Mater. in Medicine, 5, 33 (1994)) synthesized similar poly(ether-ester)s by a simple ring-opening mechanism. Findings of cytotoxicity and hemocompatibility tests showed biocompatibility. Lee and partners (J. Control. Release, 73, 315 (2001)) reported amphiphilic block copolymeric micellar systems composed of methoxy poly(ethylene glycol)/epsilon-caprolactone for DDS. Cohn et al (J. Biomed. Mater. Res. 59, 273 (2002)) produced series of PEG-PCL-containing biodegradable poly(ether-ester-urethane)s, covering a wide range of compositions. Finally, reduction of adhesions associated with post-operative surgery based on the administration of polymeric composition comprising chain-extended poly(hydroxy-carboxylic acid)/poly(oxyalkylene) ABA triblocks to a site in the body which has been subjected to trauma, e.g. by surgery, excision or inflammatory disease was described (Cohn et al. in U.S. Pat. Nos. 5,711,958 and 6,136,333).

Unfortunately, the few absorbable polymers clinically available today are stiff solids which are, therefore, clearly unsuitable for non-invasive surgical procedures, where injectability is a fundamental requirement. The only way to avoid the surgical procedure with these polymers, is to inject them as micro or nanoparticles or capsules, typically containing a drug to be released. As an example, injectable implants comprising calcium phosphate particles in aqueous viscous polymeric gels, were first proposed by Wallace et al. in U.S. Pat. No. 5,204,382. Even though the ceramic component is generally considered to be nontoxic, the use of nonabsorbable particulate material seems to trigger a foreign body response both at the site of implantation as well as at remote sites, due to the migration of the particles, over time.

Other approaches aiming at developing polymers for non-invasive techniques were intended. Among them, the use of thermosensitive gels is remarkable. The gels can be classified into two categories: (a) if they have an upper critical solution temperature (UCST), they are named positive-sensitive hydrogels and they contract upon cooling below the UCST, or (b) if they have a lower critical solution temperature (LCST), they are called negative-sensitive hydrogels and they contract upon heating above this temperature. The reverse thermo-responsive phenomenon is usually known as Reversed Thermal Gelation (RTG) and it constitutes one of the most promising strategies for the development of injectable systems. The water solutions of these materials display low viscosity at ambient temperature, and exhibit a sharp viscosity increase as temperature rises within a very narrow temperature interval, producing a semi-solid gel once they reach body temperature. There are several known RTG displaying polymers. Between them, poly(N-isopropyl acrylamide) (PNIPAAm) (Tanaka and co-workers in U.S. Pat. No. 5,403,893 and Hoffman A. S. et al., J. Controlled Release, 297, 6 (1987)), PEG-PLGA-PEG triblock polymers (Jeong et al., Nature, 388, 860-2 (1997)), etc. Unfortunately, poly(N-isopropyl acrylamide) is non-biodegradable and, in consequence, is not suitable for a diversity of applications where biodegradability is required. One of the most important RTG-displaying materials is the family of poly(ethylene oxide)/poly(propylene oxide)/poly(ethylene oxide) (PEO-PPO-PEO) triblocks, available commercially as Pluronic^{RTM} (Krezanoski in U.S. Pat. No. 4,188,373). Another known system which is liquid at room temperature, and becomes a semi-solid when warmed to about body temperature, is disclosed in U.S. Pat. No. 5,252,318, and consists of tetrafunctional block polymers of polyoxyethylene and polyoxypropylene condensed with ethylenediamine (commercially available as Tetronic.^{RTM}). Even though these materials exhibit a significant increase in viscosity when heated up to 37° C., the levels of viscosity attained are not high enough for most clinical applications. Derived from this fundamental limitation, these systems display unsatisfactory mechanical properties and unacceptably short residence times at the implantation site. Furthermore, due to these characteristics, these gels have high permeabilities, a property which renders them unsuitable for drug delivery applications because of the fast drug release kinetics of these gels. Despite of their clinical potential, these materials have failed to be used successfully in the clinic, because of serious performance limitations (Steinleitner et al., Obstetrics and Gynecology, 77, 48 (1991) and Esposito et al., Int. J. Pharm. 142, 9 (1996)).

The in situ precipitation technique developed by R. Dunn, as disclosed in U.S. Pat. No. 4,938,763, is another strategy worth mentioning. These systems comprise a water soluble organic solvent, in which the polymer is soluble. Once the system is injected, the organic solvent gradually dissolves in the aqueous biological medium, leaving behind an increasingly concentrated polymer solution, until the polymer precipitates, generating the solid implant in situ. A similar approach has been reported by Kost et al. (J. Biomed. Mater. Res., 50, 388-396 (2000)).

Additionally, in situ polymerization and/or crosslinking is another important technique used to generate injectable polymeric systems. Hubbell et al. described in U.S. Pat. No. 5,410,016, water soluble low molecular precursors having at least two polymerizable groups, that are syringed into the site and then polymerized and/or crosslinked in situ chemically or preferably by exposing the system to UV or visible radiation. Mikos et al. (Biomaterials, 21, 2405-2412 (2000)) described similar systems, whereas Langer et al. (Biomaterials, 21, 259-265 (2000)) developed injectable polymeric systems based on the percutaneous polymerization of precursors, using UV radiation.

An additional approach was disclosed by Scopelianos and co-workers in U.S. Pat. No. 5,824,333 based on the injection of hydrophobic bioabsorbable liquid copolymers, suitable for use in soft tissue repair.

Unfortunately, all these techniques have serious drawbacks and limitations, which significantly restrict their applicability. The paradox in this area has to do, therefore, with the large gap existing between the steadily increasing clinical demand for injectables, on one hand, and the paucity of materials suitable to address that need, on the other hand.

The sol-gel process whereby inorganic networks are formed from silicon or metal alkoxide monomer precursors is broadly used in diverse areas, including the glass and ceramic fields. Typically, three reactions are involved in the sol-gel process, namely hydrolysis, alcohol condensation, and water condensation. One of the main advantages of this method, is that homogeneous inorganic oxide materials with valuable properties such as chemical durability, hardness, optical transparency, appropriate porosity and thermal resistance, can be produced at room temperature. This, as opposed to the much higher temperatures required in the production of conventional inorganic glasses.

The most widely used materials are alkoxysilanes such as tetramethoxysilane (TMOS) and tetraethoxysilane (TEOS). A number of factors will significantly affect the characteristics and properties of a particular sol-gel inorganic network. Specially important are temperature and pH, on one hand, and the type and concentration of the catalyst and the water/silicon molar ratio.

The hydrolysis of the alkoxide groups (OR) results in their replacement with hydroxyl moieties (OH). The subsequent condensation reaction involving the silanol groups (Si—OH) produces siloxane bonds (Si—O—Si) plus the by-products water or alcohol. The relative rate of the hydrolysis and condensation reactions is such that, under most conditions, the latter starts before the former is complete. By fine tuning various experimental parameters such as the pH of the system, the H₂O/Si molar ratio and the type of catalyst, the hydrolysis reaction can be brought to completion before the condensation step starts.

The polymerization process can be conducted in three different pH regions: below pH=2, between pH=2 and pH=7 and for pH values higher than 7. The overall process occurs in three stages: (i) First, particles form due to the polymerization of the precursors; (ii) Then, the particles grow and finally (iii) The particles join forming chains and then networks that extend throughout the liquid medium, thickening into a gel (R. K. Iler, The Chemistry of Silica, Wiley: New York, 1979).

It has been observed that the rate and extent of the hydrolysis reaction is largely influenced by both the strength and the concentration of the acid or base catalyst. It has been reported that the reaction is faster for pH values below 5 or, alternatively, above 7 (Weiss P. et al., Biopolymers, 63, 232-238 (2002)). Expectedly, larger H₂O/Si molar ratios normally encourage hydrolysis. It should be also stressed that, since water is the by-product of the condensation reaction, large water contents promote siloxane bond hydrolysis.

The pH of the medium plays an important role also during the condensation stage. At pH values between 6 and 7 the reaction is at its lowest pace, while in the 2-6 pH range and above pH=7 the reaction is faster. In addition, even though the condensation stage can proceed without catalyst, the use of a catalyst is helpful. The acid-catalyzed condensation mechanism involves the protonation of the silanol species, as a result of which the silicon becomes more electrophilic and, thus, more susceptible to nucleophilic attack. The most widely accepted mechanism for the base-catalyzed condensation reaction involves the attack of a nucleophilic deprotonated silanol on a neutral silicic acid (R. K. Iler, The Chemistry of Silica, Wiley: New York, 1979).

As to the structure of the materials obtained, it can be stated that when the reaction is performed under acidic conditions, the sol-gel derived silicon oxide networks primarily comprise linear or randomly branched polymers which, in turn, entangle and form additional branches resulting in gelation. On the other hand, silicon oxide networks obtained under base-catalyzed conditions produce more highly branched clusters which do not interpenetrate prior to gelation and thus behave as discrete clusters.

Some work has been conducted aiming at developing inorganic-organic telechelic polymers. For example, Bunel et al. (Polymer, 39, 965-971 and 973-979 (1998)) described the functionalization of low molecular weight polybutadiene chains with triethoxysilane and their crosslinking at temperatures ranging from 20° C. to 80° C. for 30 days. Seppala and co-workers (Polymer, 42, 3345-3353 (2001)) reported the modification of polylactic acid. The crosslinking was carried out at drastic conditions: 60° C.-120° C. in presence of nitric acid as the catalyst. Osaka and collaborators (J. Sol-Gel Sci. Tech., 21, 115-121 (2001) prepared hybrid materials incorporating gelatin and 3-(glycidoxypropyl) trimethoxysilane through sol-gel processing. Zhu and co-workers (J. Mat. Sc. Mat. Med., 14, 27-31 (2003)) prepared silica-butyrylchitosan hybrid films, using butyrylchitosan as the organic species incorporated into the system. The sol-gel process was carried out in hydrochloric acid and methanol at RT for several days and heating at 80° C. for 2 hours.

The use of silica to induce the formation and deposition of calcium phosphate (CaP) derivatives such as apatite and hydroxyapatite for bone regeneration, was studied. Thus, Li and collaborators (J. Non-Cryst. Sol., 168, 281-286 (1994) and J. Biomed. Mat. Res., 29, 325-328 (1995)) reported that silica gels sintered at 900-1000° C. can stimulate apatite crystallization from metastable calcium phosphate solutions on their surfaces. Pereira and Hench (J. Sol-Gel Sci. Tech., 7, 59-68 (1996)) studied the mechanism of hydroxyapatite formation onto porous silica substrates. Canham et al. (Thin Solid Films, 297, 304-307 (1997)) investigated the nucleation of calcium phosphate on porous silicon in the presence of simulated body fluids (SBF). Varma and co-workers (J. Mat. Sci. Mat. Med., 12, 767-773 (2001)) functionalized cotton fibers with tetraethoxysilane and studied the growth of CaP on it. Lopatin et al. (J. Mat. Sci. Mat. Med., 12, 767-773 (2001)) used silicon substrates to grow HA and tricalcium phosphate. Reis and co-workers (J. Mat. Sci. Mat. Med., 13, 1181-1188 (2002)) used starch based biomaterials coated with sodium silicate for the cell adhesion and proliferation on biomimetic CaP. Finally, Nakamura and his group (Biomaterials, 24, 1349-1356 (2003)) developed calcium oxide-containing glasses and evaluated the apatite formation in contact with simulated body fluid solution.

The present invention capitalizes on the advantages of modified polymers displaying low viscosities at deployment time via minimally or non-invasive surgical procedures, and which contain mono, bi or trifunctional silicone-containing reactive groups, most importantly alkoxysilane or silanol groups, capable of undergoing a hydrolysis-condensation reaction at a predetermined body site, in the physiological conditions of humidity and temperature, whereby their molecular weight increases as a result of their polymerization and/or crosslinking, to render in situ generated implants of appropriate and advantageous properties.

According to the present invention an environmentally responsive polymeric system comprising a polymeric component containing reactive Si-based moieties capable of generating stable and inert Si—O—Si bonds primarily at a predetermined body site is now provided, as a result of which the molecular weight of the polymeric system increases and a change in its rheological and mechanical properties is produced. In some instances, these materials generate silicon-rich domains.

More specifically the present invention provides a responsive polymeric system comprising one or more silicon-containing reactive groups capable of undergoing a condensation reaction effected primarily at a predetermined body site in the presence of water and at body temperature wherein said reaction results in an increase in the molecular weight of the polymeric system due to polymerization and/or crosslinking and produces at least a partial change in the rheological and mechanical properties of said system.

In a preferred embodiment of the present invention said responsive polymeric system is deployable at a predetermined body site via a non-invasive or a minimally invasive surgical procedure.

In a preferred embodiment of the present invention said responsive polymeric system is biodegradable whereby the system disappears from the site due to chain scission, decrease in molecular weight and final solubilization into the aqueous environment, or said system is selectively biodegradable whereby the chain scission phenomenon is confined to sections of the polymerized and/or crosslinked polymer, so that said polymer reverts to an oligomer or to essentially un-polymerized or non-crosslinked state.

In said preferred embodiments the responsive polymeric system may generate a linear polymer, a block polymer, a graft polymer, a comb polymer, a star-like polymer, a crosslinked polymer and combinations thereof.

In said preferred embodiments, the responsive polymeric system preferably comprises also additional reactive groups such as hydroxyl, carboxyl, thiol, amine, isocyanate, thioisocyanate capable of cross-linking reaction, or unsaturated moieties capable of polymerizing by an addition polymerization mechanism such as a free radical polymerization, yielding different Interpenetrating Polymer Networks (IPN) or semi-Interpenetrating Polymer Networks and combinations thereof. An Interpenetrating Polymer Network comprises at least two cross-linked polymers blended at a molecular level, with no covalent bonds connecting between the two, and a semi-Interpenetrating Polymer Network comprises one cross-linked polymer embedded by a non-crosslinked one, with no covalent bonds connecting between the two.

In said preferred embodiments preferably the responsive polymeric system comprises more than one component that form covalent bonds between them or generate physical blends or interpenetrating or pseudo-interpenetrating networks and combinations thereof, at the predetermined body site.

In further preferred embodiments preferably the responsive polymeric system contains biomolecule/s to be delivered into the body such as drugs, oligopeptides, peptides, growth factors, enzymes, hormones, elastin, collagenous material, albumin, a fibrinous material, living cells such as endothelial cells, hepatocytes, smooth muscle cells, bone marrow cells, astrocytes, osteoblasts, chondrocytes, fibroblasts, myocytes, materials of tissue origin such as demineralized tissue or an acellular tissue matrix and combinations thereof.

In said more preferred embodiments the responsive polymeric system may comprise also a macro, micro or nano-sized solid component such as a polymer, a ceramic material, a metal, a carbon, a biological material, and combinations thereof, presenting the solid component different and various shapes such as particles, spheres, capsules, rods, slabs, fibers, meshes, ribbons, webs, non-woven structures, fabrics, amorphous lattice structures, filament wound structures, honeycomb or braided structures, and combinations thereof, wherein said solid component may be hollow, porous or solid, and combinations thereof.

In said more preferred embodiments the solid component possesses reactive moieties capable of reacting with the silicon-containing reactive groups present in said responsive polymeric system or with any other components present in the system such as those generating an IPN or Semi-IPN with said silicon-containing polymer.

In another preferred embodiments the solid component is a ceramic material selected from a group consisting of tricalcium phosphate or hydroxyapatite and combinations thereof.

In even more preferred embodiments said silicon-containing responsive polymeric system is a low molecular weight polymer capable of being deployed at a predetermined body site by minimally invasive procedures, such as polyoxyalkylene, polyester, polyurethane, polyamide, polycarbonate, polyanhydride, polyorthoesters, polyurea, polypeptide, polyalkylene, acrylic or methacrylic polymers, polysaccharide and combinations thereof.

In even more preferred embodiments the responsive polymeric system is also capable of undergoing a transition that results in a sharp increase in viscosity in response to a predetermined trigger such as temperature, pH, ionic strength, at a predetermined body site, resulting in an increase in the viscosity of said responsive polymeric system by at least about 2 times, wherein said transition takes place before and/or during and/or after the chemical triggering reaction.

In even more preferred embodiments the responsive polymeric system comprises water or an aqueous-based solvent.

In especially preferred embodiments the responsive polymeric system is a polyoxyalkylene polymer, a block copolymer comprising polyethylene oxide (PEO) and polypropylene oxide (PPO) selected from a group consisting of a diblock, a triblock or a multiblock, a segmented block copolymer comprising polyethylene oxide (PEO) and polypropylene oxide (PPO) chains, wherein said PEO and PPO chains are connected via a chain extender, a poly(alkyl-co-oxyalkylene) copolymer having the formula R—(OCH₂CH)ₙ—OH, where R is an hydrophobic monofunctional segment selected from a group consisting of poly(tetramethylene glycol), poly(caprolactone), poly(lactic acid), poly(siloxane) and combinations thereof, a poly(alkyl-co-oxyalkylene) copolymer having the formula [—R′—(OCH₂CH)ₙ—O]ₚH, where R′ is a bifunctional or multifunctional hydrophobic segment, a poly(N-alkyl substituted acrylamide), preferably poly(N-isopropyl acrylamide), cellulose and cellulose derivatives, alginates and its derivatives, hyaluronic acid and its derivatives, collagen, gelatin, chitosan and its derivatives, agarose, water soluble synthetic, semi-synthetic or natural oligomers and polymers selected from a groups consisting of oligoHEMA, polyacrylic acid, polyvinyl alcohol, polyethylene oxide, TMPO, oligo and polysaccharides, oligopeptides, peptides, proteins, and combinations thereof.

Preferably said chain extender comprises phosgene, aliphatic or aromatic dicarboxylic acids or their reactive derivatives such as acyl chlorides and anhydrides or other molecules able to react with the OH terminal groups of the PEO and PPO chains, such as dicyclohexylcarbodiimide (DCC), aliphatic or aromatic diisocyanates such as hexamethylene diisocyanate (HDI) or methylene bisphenyldiisocyanate (MDI) or cyanuric chloride or any other bifunctional or multifunctional segment, and/or combinations thereof.

In even more preferred embodiments the responsive polymeric system contains other polymers that are responsive to other stimuli selected from a group consisting of temperature, pH, ionic strength, electric and magnetic fields, energy sources covering a broad range of wavelengths such as ultraviolet, visible, infrared, microwave, ultrasound, electron beam and x-rays radiation, fluids and biological species, and combinations thereof.

Preferably said responsive component contains biologically or pharmacologically active molecule/s, to be delivered into the body following a unimodal or multimodal time dependent release kinetics, as the molecular weight of the polymeric system as well as its rheological and mechanical properties change at the predetermined body site.

In said preferred embodiments said biologically or pharmacologically active molecule/s to be delivered into the body are covalently bound to said silicon-containing responsive polymer or any other component of the system via biodegradable spacers, rendering homogeneously distributed reservoirs.

In even more preferred embodiments the responsive component can be used as sealants, as coatings and lubricants, as transient barriers for the prevention of post-surgical adhesions, as matrices for the unimodal or multimodal controlled release of biologically active agents, in the area of Tissue Engineering and the field of Gene Therapy.

In further preferred embodiments the silicon moieties serve as nuclei for the deposition or crystallization of various materials preferably hydroxyapatite or other calcium phosphate derivatives for bone regeneration induction at a predetermined body site.

The novel, tailor-made compositions of the present invention display advantageous properties unattainable by the prior art by capitalizing, in a unique and advantageous way, on the low viscosity of the silicon-containing reactive groups polymeric system at the insertion/administration time, and the molecular weight increase and/or crosslinking in situ, with or without additional additives or initiator/catalyst systems.

Compositions according to this invention are suitable to be used in the human body, preferably in applications where the combination of ease of insertion and enhanced initial flowability, on one hand, and post-implantation high viscosity and superior mechanical properties, on the other hand, are required.

Aiming to expand the clinical applicability of the biomedical hydrogels, it is an object of this invention to provide superior responsive polymeric systems. These materials will find a variety of important biomedical applications in the biomedical field, such as in non-invasive surgical procedures, in drug delivery systems, in the prevention of post-surgical adhesions and in the Tissue Engineering field, designed to cover a broad range of mechanical properties. In the case of biodegradable systems, these materials are engineered to display different degradation kinetics.

It is an additional object of the invention to introduce hydrolytically unstable segments along the polymeric backbone, allowing to fine tune both the degradation rate of the polymer molecule as well as to control the stability of the whole system and its rheological properties. It is an additional object of the invention to render these compositions with specific biological functions by incorporating biomolecules of various types, physically (by blending them into the system) or chemically (by covalently binding them to the polymer). It is an additional object of the invention to incorporate cells of various types into these materials, for them to perform as RTG-displaying matrices for cell growth and tissue scaffolding.

While the invention will now be described in connection with certain preferred embodiments in the following examples and with reference to the appended Figures, so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

In the drawings:

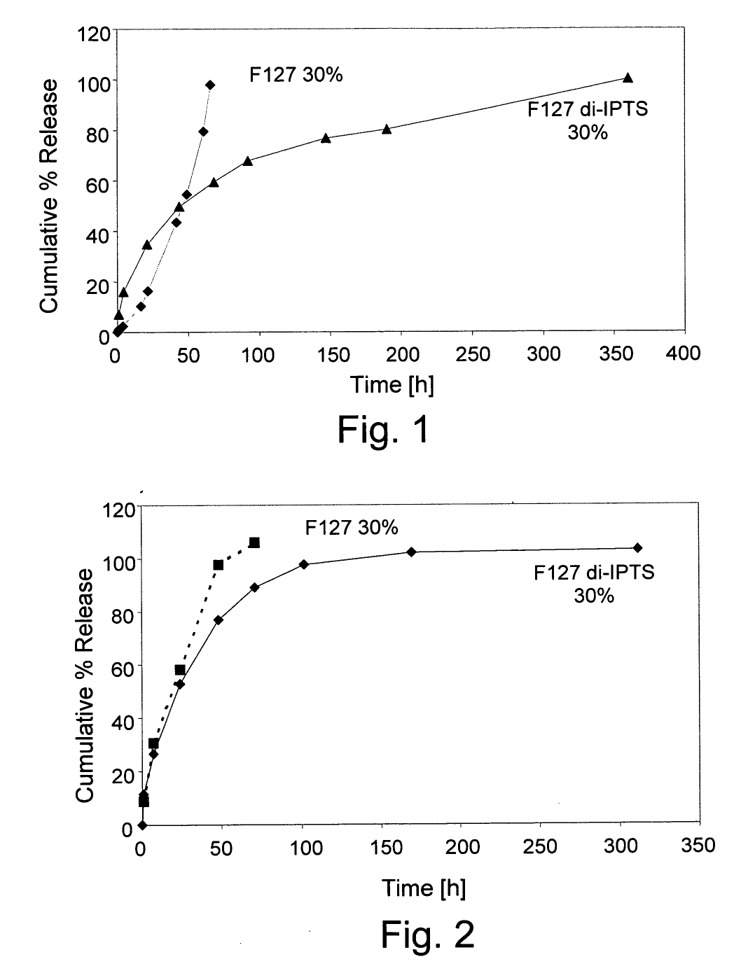

FIG. 1 is a graphic representation of the release of Methylene Blue from F127 di-IPTS 30% in comparison to Pluronic F127 30% gel;

FIG. 2 is a graphic representation of the release of Metronidazole from F127 di-IPTS 30% in comparison to Pluronic F127 30% gel;

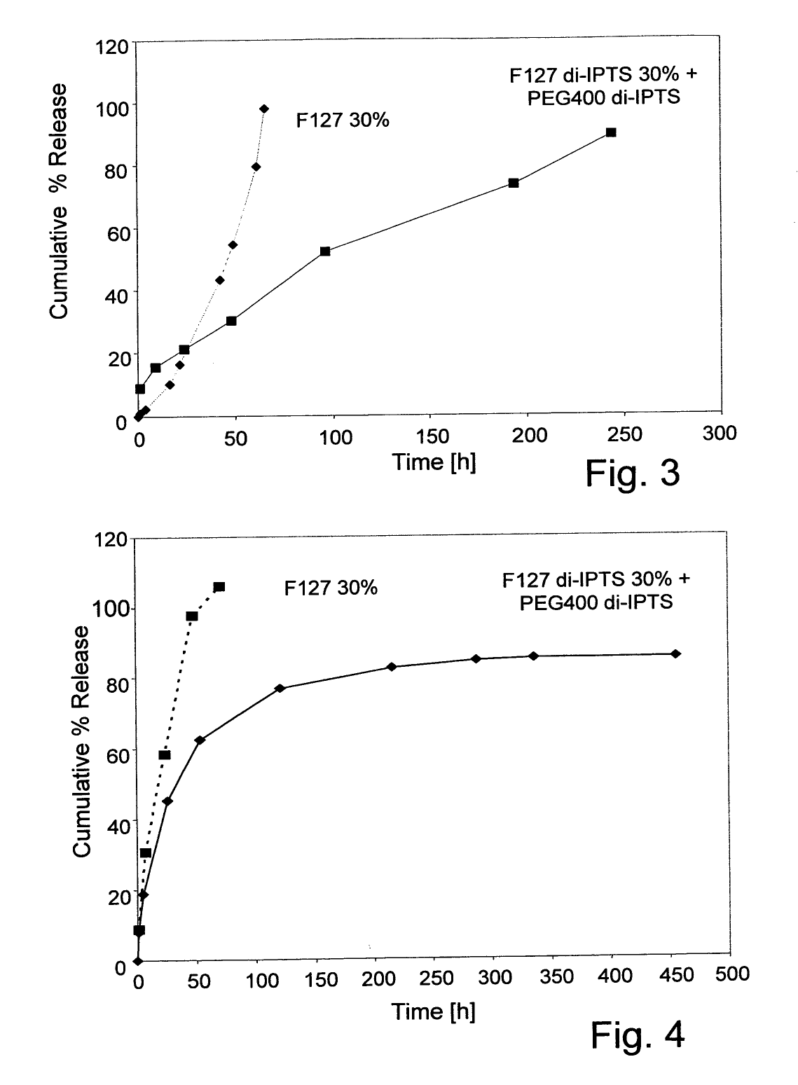

FIG. 3 is a graphic representation of the release of methylene blue from a F127 di-IPTS/PEG400 di-IPTS in comparison to Pluronic F127 30% gel;

FIG. 4 is a graphic representation of the release of Metronidazole from a F127 di-IPTS/PEG400 di-IPTS co-hydrogel in comparison to Pluronic F127 30% gel;

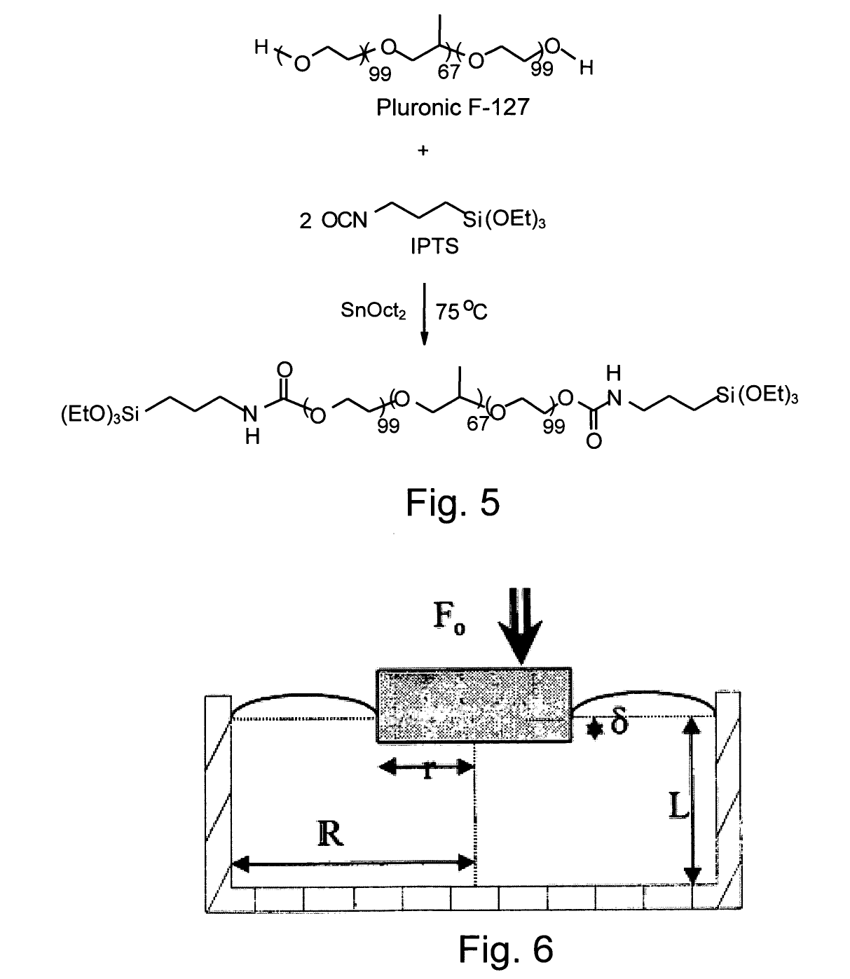

FIG. 5 presents reaction Scheme 1 of synthesis of F127 di-IPTS;

FIG. 6 is the measuring device in the compression test taken from Gregson et al., Carbohydrate Polymers, 38, 255-259 (1999);

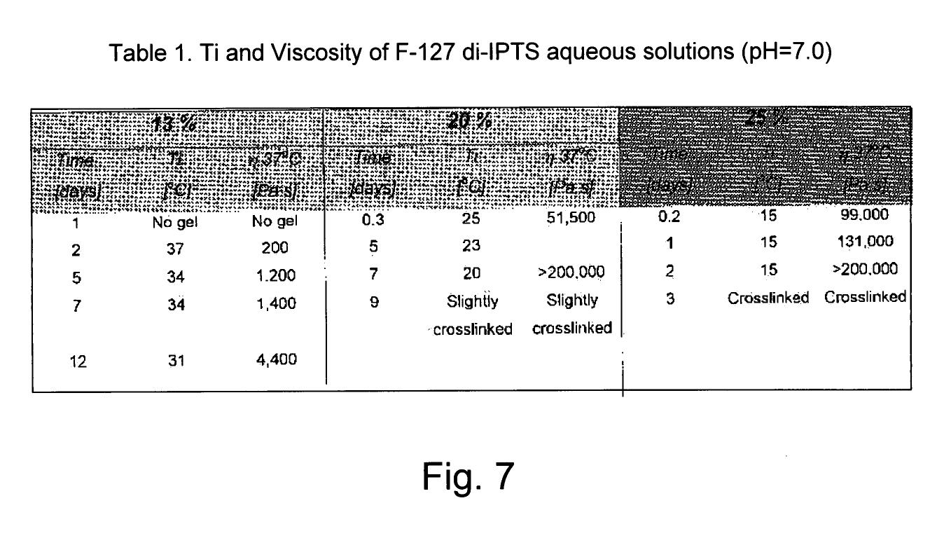

FIG. 7 presents Table 1;

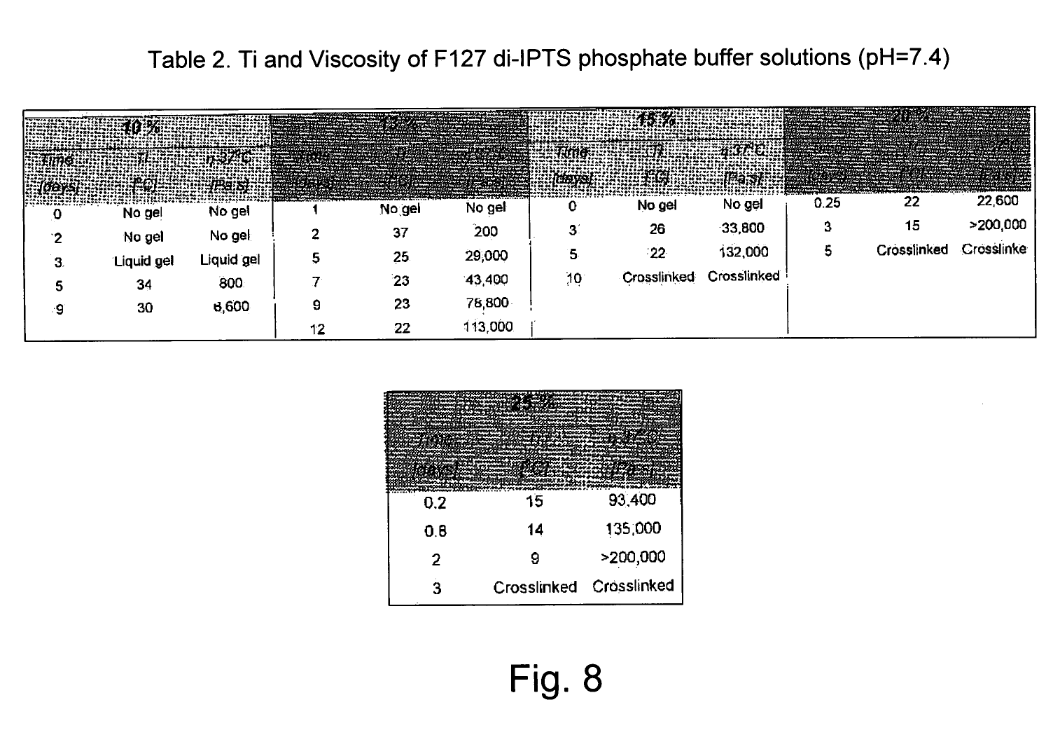

FIG. 8 presents Table 2; and

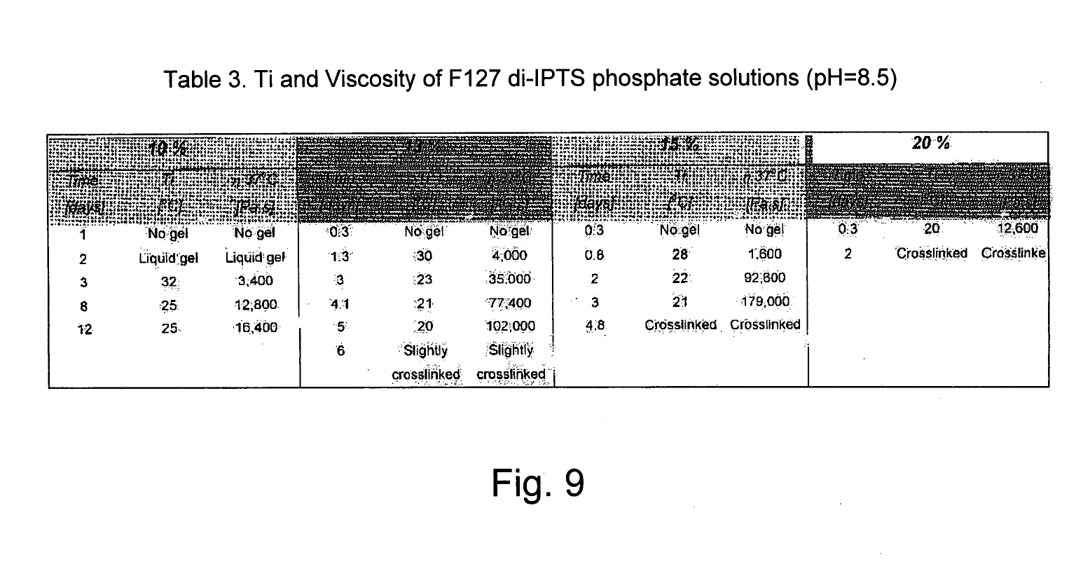

FIG. 9 presents Table 3.

### EXAMPLES

### Example 1

### Pluronic F127 di-(3-isocyanatopropyl)triethoxysilane (F127 di-IPTS)

a) Synthesis of F127 di-IPTS

25.2 g (0.002 mol) Pluronic F127 (molecular weight 12,600) were poured in a three-necked flask and dried at 120° C. under vacuum for 2 hours. Then, 1.2 g (0.005 mol) IPTS and 0.1 g (3.10⁻⁴ mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in petroleum ether 40-60 (400 ml). Finally, the F127 derivative was washed repeatedly with portions of petroleum ether and dried in vacuum at RT. The synthesis is presented in Scheme 1 (see FIG. 5).

b) Polymerization of F127 di-IPTS

F127 di-IPTS was dissolved in water-based solvent in different concentrations and the solutions were incubated at 37° C. The polymerization process includes two stages. The first comprises the ethoxysilane group hydrolysis to silanol groups and the second the condensation of the generated silanol groups to form Si—O—Si bonds.

c) Rheological Behavior of F127 di-IPTS Solutions in Water at 37° C. with Time

The viscosity and the gelation temperature (Tᵢ) at 37° C. for 13, 20 and 25% solutions in water (pH about 7) were measured vs. time and are presented in Table 1 (see FIG. 7).

d) Rheological Behavior of F127 di-IPTS solutions in PBS (pH=7.4) at 37° C. with Time

The viscosity and the T_{I} at 37° C. for 10, 13, 15, 20 and 25% solutions in PBS (pH=7.4) were measured vs. time and are presented in Table 2 (FIG. 8).

e) Rheological Behavior of F127 di-IPTS Solutions in PBS (PH=8.5) at 37° C. with Time

The viscosity and the T_{I} at 37° C. for 10, 13, 15, and 20 solutions in PBS adjusted to pH 8.5 with NaOH were measured vs. time and are presented in Table 3 (see FIG. 9).

f) Preparation of F127 di-IPTS Containing 50 mg Methylene Blue/5 g Gel

50 mg methylene blue were added to 5 g of a 30% F127 di-IPTS solution in water and the system was incubated at 37° C. for 96 h. The same was done with 30% F127 for comparison.

g) Release of Methylene Blue from a F127 di-IPTS Gel

20 ml PBS 0.1 M were added to each of the gels prepared in f) and incubated in a Gyratoty Water Bath Shaker at 37° C. for different periods of time. The supernatant solution was renewed periodically and the absorbance of the methylene blue solution was determined at 664 nm. The release was expressed as cumulative % released and compared to the release of a 30% F127 gel (see FIG. 1).

h) Preparation of F127 di-IPTS Blend Containing 25 mg Metronidazole/5 g Gel

25 mg metronidazole were added to 5 g of a 30% F127 di-IPTS solution in and the system was incubated at 37° C. for 96 h. The same was done with 30% F127 for comparison.

i) Release of Metronidazole from a F127 di-IPTS Gel

20 ml PBS 0.1 M were added to each of the gels prepared in h) and incubated in a Gyratoty Water Bath Shaker at 37° C. for different periods of time. The supernatant solution was renewed periodically and the absorbance of the metronidazole solution was determined at 319 nm. The release was expressed as cumulative % released (see FIG. 2).

### Example 2

### Pluronic F38 di-(3-isocyanatopropyl)triethoxysilane (F38 di-IPTS)

a) Synthesis of F38 di-IPTS

20.1 g (0.004 mol) Pluronic F38 (molecular weight 4,600) were poured in a three-necked flask and dried at 120° C. under vacuum for 2 hours. Then, 2.6 g (0.01 mol) IPTS and 0.2 g (3.10⁻⁴ mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in petroleum ether 40-60 (400 ml). Finally, the F38 derivative was washed repeatedly with portions of petroleum ether and dried in vacuum at RT.

b) Polymerization of F38 di-IPTS

A 40% F38 di-IPTS solution in PBS was incubated at 37° C. to obtain a crosslinked gel.

### Example 3

### Poly(ethylene glycol) MW=400 di-(3-isocyanatopropyl)triethoxysilane (PEG400 di-IPTS)

5.1 g (0.013 mol) PEG400 were poured in a three-necked flask and dried at 120° C. under vacuum for 1 hours. Then, 7.6 g (0.019 mol) IPTS and 1.5 g (0.004 mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in petroleum ether 40-60 (400 ml). Finally, the PEG400 di-IPTS was washed repeatedly with portions of petroleum ether and dried in vacuum at RT. Whereas the material was a liquid at 37° C., after incubation at this temperature a brittle and transparent film was formed.

### Example 4

### Poly(ethylene glycol) MW=600 di-(3-isocyanatopropyl)triethoxysilane (PEG600 di-IPTS)

20.1 g (0.034 mol) PEG600 were poured in a three-necked flask and dried at 120° C. under vacuum for 1 hours. Then, 18.3 g (0.007 mol) IPTS and 1.5 g (0.004 mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in petroleum ether 40-60 (400 ml). Finally, the PEG600 di-IPTS was washed repeatedly with portions of petroleum ether and dried in vacuum at RT. Whereas the material was a liquid at 37° C., after incubation at this temperature a brittle and transparent film was formed.

### Example 5

### Poly(ethylene glycol) MW=1000 di-(3-isocyanatopropyl)triethoxysilane (PEG1000 di-IPTS)

10.2 g (0.010 mol) PEG1000 were poured in a three-necked flask and dried at 120° C. under vacuum for 1 hours. Then, 5.4 g (0.022 mol) IPTS and 0.5 g (0.001 mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in petroleum ether 40-60 (400 ml). Finally, the PEG1000 di-IPTS was washed repeatedly with portions of petroleum ether and dried in vacuum at RT. Whereas the material was a paste at 37° C., after incubation at this temperature a brittle and transparent film was formed.

### Example 6

### Pluronic F127 di-(3-isocyanatopropyl)triethoxysilane/Poly(ethylene glycol) MW=400 di-(3-isocyanatopropyl)triethoxysilane (PEG400 di-IPTS) co-hydrogel Containing Methylene Blue

a) Synthesis of F127 di-IPTS

The synthesis of F127 di-IPTS was described in Example 1a.

b) Synthesis of PEG400 di-IPTS

The synthesis of PEG400 di-IPTS was described in Example 3.

c) Preparation of F127 di-IPTS/PEG400 di-IPTS Co-Hydrogel Containing 50 mg Methylene Blue/5 g gel

0.1 g (0.0001 mol) PEG400 di-IPTS were added to 5 g of a 30% F127 di-IPTS solution in water. Then, 50 mg methylene blue were dissolved and the system was incubated at 37° C. for 96 h.

d) Release of Methylene Blue from a F127 di-IPTS/PEG400 di-IPTS Co-Hydrogel

20 ml PBS 0.1 M were added to the gel prepared in c) and incubated in a Gyratoty Water Bath Shaker at 37° C. for different periods of time. The supernatant solution was renewed periodically and the absorbance of the methylene blue solution was determined at 664 nm. The release was expressed as cumulative % released and was compared to the release of 30% F127 gel (see FIG. 3).

### Example 7

### Pluronic F127 di-(3-isocyanatopropyl)triethoxysilane/Poly(ethylene glycol) MW=400 di-(3-isocyanatopropyl)triethoxysilane (PEG400 di-IPTS) Co-Hydrogel Containing Metronidazole

a) Synthesis of F127 di-IPTS

The synthesis of F127 di-IPTS was described in Example 1a).

b) Synthesis of PEG400 di-IPTS

The synthesis of PEG400 di-IPTS was described in Example 3.

c) Preparation of F127 di-IPTS/PEG400 di-IPTS co-hydrogel containing 25 mg metronidazole/5 g gel

0.1 g (0.0001 mol) PEG400 di-IPTS were added to 5 g of a 30% F127 di-IPTS solution in water. Then, 25 mg metronidazole were dissolved and the system was incubated at 37° C. for 96 h.

d) Release of Metronidazole from a F127 di-IPTS/PEG400 di-IPTS Co-Hydrogel

20 ml PBS 0.1 M were added to the gel prepared in c) and incubated in a Gyratoty Water Bath Shaker at 37° C. for different periods of time. The supernatant solution was renewed periodically and the absorbance of the metronidazole solution was determined at 319 nm. The release was expressed as cumulative % released and was compared to the release of 30% F127 gel (see FIG. 4).

### Example 8

### (Polycaprolactone)₄-Poly(ethyleneglycol)-(Polycaprolactone)₄ ((CL)₄-PEG1000-(CL)₄) di-(3-isocyanatopropyl)triethoxysilane

a) Synthesis of the (CL)₄-PEG1000-(CL)₄ Triblock

The (CL)₄-PEG1000-(CL)₄ triblock was synthesized by a typically ring-opening polymerization reaction as follows: 30.2 g (0.03 mol) of dry PEG1000 (1 h at 120° C. in vacuum) and 32.8 g (0.3 mol) of epsilon-caprolactone (20% in molar excess) were reacted in a 100 ml flask in presence of SnOct₂ (0.3 g) at 145-150° C., in a dry N₂ atmosphere and with magnetic stirring. The reaction was continued for 2.5 hours. The material was a wax at RT.

b) Synthesis of (CL)₄-PEG1000-(CL)₄ di-(3-isocyanatopropyl)triethoxysilane

19.8 g of the triblock prepared in a) were dried for 1 h at 120° C. in vacuum. Then the temperature was stabilized at 80° C. and 0.5 g catalyst and 5.9 g (0.024 mol) IPTS were added. The reaction continued for 1 h at this temperature in dry N₂ atmosphere. Finally, the reaction mixture was cooled to RT, washed with 50 ml of petroleum ether 40-60 and dried at RT in vacuum for 24 hours. The material was a yellow paste at RT.

c) Crosslinking of (CL)₄-PEG1000-(CL)₄ di-IPTS

5 g of (CL)₄-PEG1000-(CL)₄ di-IPTS synthesized in b) were poured in a 25 ml vial (30 mm diameter) and heated at 37° C. Then 1 ml PBS (pH 7.4 0.1 M) was added onto the material. The system was incubated at 37° C. The resulting material was yellow and transparent.

### Example 9

### (Polycaprolactone)₆-Poly(ethyleneglycol)1000-(Polycaprolactone)₆ di-(3-isocyanatopropyl)triethoxysilane ((CL)₆-PEG1000-(CL)₆ di-IPTS)

a) Synthesis of the (CL)₆-PEG1000-(CL)₆ Triblock

30.1 g (0.03 mol) of dry PEG1000 (1 h at 120° C. in vacuum) and 49.3 g (0.43 mol) of epsilon-caprolactone (20% in molar excess) were reacted in a 100 ml flask in presence of SnOct₂ (0.45 g) at 145-150° C., in a dry N₂ atmosphere and with magnetic stirring. The reaction was continued for 2.5 hours. The material was a wax at RT.

b) Synthesis of (CL)₆-PEG1000-(CL)₆ di-(3-isocyanatopropyl)triethoxysilane

20.2 g of the triblock prepared in a) were dried for 1 h at 120° C. in vacuum. Then the temperature was stabilized at 80° C. and 0.42 g catalyst and 5.1 g (0.02 mol) IPTS were added. The reaction continued for 1 h at this temperature in N₂ atmosphere. Finally, the reaction mixture was cooled to RT, washed with 50 ml of petroleum ether 40-60 and dried at RT in vacuum for 24 hours. The material was a yellow paste at RT.

c) Crosslinking of (CL)₆-PEG1000-(CL)₆ di-IPTS

5 g of (CL)₆-PEG1000-(CL)₆ di-IPTS synthesized in b) were poured in a 25 ml vial (30 mm diameter) and heated at 37° C. Then 1 ml PBS (pH 7.4 0.1 M) was added onto the material. The system was incubated at 37° C. The resulting material was yellow and transparent.

### Example 10

### Polycaprolactone MW=530 di-(3-isocyanatopropyl)triethoxysilane (PCL530 di-IPTS)

a) Synthesis of PCL530 di-IPTS

20.2 g of PCL530 were dried for 1 h at 120° C. in vacuum. Then the temperature was stabilized at 80° C. and 1.9 g catalyst and 22.4 g (0.09 mol) IPTS were added. The reaction continued for 1 h at this temperature in N₂ atmosphere. Finally, the reaction mixture was cooled to RT, washed with 50 ml of petroleum ether 40-60 and dried at RT in vacuum for 24 hours. The material was a slightly yellow liquid at RT.

b) Crosslinking of PCL530 di-IPTS

5 g of PCL530 di-IPTS synthesized in a) were poured in a 25 ml vial (30 mm diameter) and heated at 37° C. Then 1 ml PBS (pH 7.4 0.1 M) was added onto the material. The system was incubated at 37° C. The resulting material was yellow and transparent.

### Example 11

### Polycaprolactone MW=2000 di-(3-isocyanatopropyl)triethoxysilane (PCL2000 di-IPTS)

a) Synthesis of PCL2000 di-IPTS

10.2 g of PCL2000 (0.005 mol) were poured in 100 ml flask and heated to 80° C. and 0.25 g catalyst and 3.1 g (0.09 mol) IPTS were added. The reaction continued for 1 h at this temperature in dry N₂ atmosphere. Finally, the reaction mixture was cooled to RT, washed with 50 ml of petroleum ether 40-60 and dried at RT in vacuum for 24 hours. The material is a white wax at RT.

b) Crosslinking of PCL2000 di-IPTS

5 g of PCL2000 di-IPTS synthesized in a) were heated to 70° C. and poured in a 25 ml vial (30 mm diameter) and heated at 37° C. Then 1 ml PBS (pH 7.4 0.1 M) were added onto the material. The system was incubated at 37° C. The resulting material was a white and hard product.

### Example 12

### Polycaprolactone-Polytetramethylene glycol-Polycaprolactone (Terethane.RTM CL MW=2000, PTMG2000 CL) di-(3-isocyanatopropyl)triethoxysilane (PTMG2000 di-IPTS)

a) Synthesis of PTMG2000 CL di-IPTS

30.1 g of PTMG2000 CL were dried for 1 h at 120° C. in vacuum. Then the temperature was stabilized at 80° C. and 0.8 g catalyst and 8.9 g (0.04 mol) IPTS were added. The reaction continued for 1 h at this temperature. Finally, the reaction mixture was cooled to RT, washed with 50 ml of petroleum ether 40-60 and dried at RT in vacuum for 24 hours. The material was a white paste at RT.

b) Crosslinking of PTMG2000 CL di-IPTS

5 g of PTMG2000 CL di-IPTS synthesized in a) were poured in a 25 ml vial (30 mm diameter) and heated at 37° C. Then 1 ml PBS (pH 7.4 0.1 M) was added onto the material. The system was incubated at 37° C. The resulting material was yellow and transparent.

### Example 13

### Trimethylolpropane ethoxylate MW=1014 tri-(3-isocyanatopropyl)triethoxysilane (TMPE1014 tri-IPTS)

a) Synthesis of TMPE1014 tri-IPTS

5.1 g of (0.005 mol) TMPE1014 were dried for 1 h at 120° C. in vacuum. Then the temperature was stabilized at 80° C. and 0.4 g catalyst and 4.4 g (0.02 mol) IPTS were added. The reaction continued for 1 h at this temperature. Finally, the reaction mixture was cooled to RT, washed with 50 ml of petroleum ether 40-60 and dried at RT in vacuum for 24 hours. The material was a liquid at RT.

b) Crosslinking of TMPE1014 tri-IPTS

5 g of TMPE1014 tri-IPTS synthesized in a) were poured in a 25 ml vial (30 mm diameter) and heated at 37° C. Then 1 ml PBS (pH 7.4 0.1 M) were added onto the material. The system was incubated at 37° C. The resulting material was a transparent product.

### Example 14

### PCL530 di-IPTS/PCL2000 di-IPTS crosslinked copolymer

a) Synthesis of PCL530 di-IPTS

The synthesis of PCL530 di-IPTS was described in Example 10a.

b) Synthesis of PCL2000 di-IPTS

The synthesis of PCL2000 di-IPTS was described in Example 11 a).

PCL530 di-IPTS/PCL2000 di-IPTS Crosslinked Copolymer

5 g of material with different PCL530 di-IPTS/PCL2000 di-IPTS ratios were poured in a 25 ml vial (30 mm diameter) and heated at 37° C. Then 1 ml PBS (pH 7.4 0.1 M) was added onto the material. The system was incubated at 37° C.

### Example 15

### PCL530 di-IPTS Crosslinked Scaffold within Pluronic F127 Matrix

a) Synthesis of PCL530 di-IPTS

The synthesis of PCL530 di-IPTS was described in Example 10a

b) Preparation of PCL530 di-IPTS Crosslinked Scaffold within Pluronic F127 Matrix

0.8 g F127 were dissolved in 3.2 g PBS (pH=7.4, 0.1 M) at 4° C. Then, 1 g PCL530 di-IPTS were added and the mixture was homogeneized and incubated at 37° C.

### Example 16

### F127 di-IPTS/PCL530 di-IPTS Crosslinked Copolymer

a) Synthesis of F127 di-IPTS

The synthesis of F127 di-IPTS was described in Example 1a).

b) Synthesis of PCL530 di-IPTS

The synthesis of PCL530 di-IPTS was described in Example 10a.

c) Preparation of F127 di-IPTS/PCL530 di-IPTS Crosslinked Copolymer

0.8 g F127 di-IPTS were dissolved in 3.2 g PBS (pH=7.4, 0.1 M) at 4° C. Then, 1 g PCL530 di-IPTS was added and the mixture was homogenized and incubated at 37° C.

### Example 17

### PTMG2000 di-IPTS crosslinked scaffold within Pluronic F127 matrix

a) Synthesis of PTMG2000 CL di-IPTS

The synthesis of PTMG2000 CL di-IPTS was described in Example 12a.

b) Preparation of PTMG2000 CL di-IPTS Crosslinked Scaffold within Pluronic F127 Matrix

0.8 g F127 were dissolved in 3.2 g PBS (pH=7.4, 0.1 M) at 4° C. Then, 1 g PTMG2000 di-IPTS was added and the mixture was homogenized and incubated at 37° C.

### Example 18

### F127 di-IPTS/PTMG2000 CL di-IPTS Crosslinked Copolymer

a) Synthesis of F127 di-IPTS

The synthesis of F127 di-IPTS was described in Example 1a).

b) Synthesis of PTMG2000 CL di-IPTS

The synthesis of PCL530 di-IPTS was described in Example 10a).

c) Preparation of F127 di-IPTS/PTMG2000 CL di-IPTS crosslinked Copolymer

0.8 g F127 di-IPTS were dissolved in 3.2 g PBS (pH=7.4, 0.1 M) at 4° C. Then, 1 g PTMG2000 CL di-IPTS was added and the mixture was homogenized and incubated at 37° C.

### Example 19

### Compression Test of Different Crosslinked Materials

The test was carried out as described by Oakenfull et al. (Gums and Stabilisers for Food Industry 4, 231-239 Ed. G. O. Phillips, P. A. Williams, D. J. Wedlock. IRL Press, Oxford (1988)). Accordingly, the apparent modulus is determined from the curve slope. All the samples were liquids to viscous liquids at 37° C., before crosslinking.

Reference should be had to FIG. 6 which depicts the measuring device (taken from Gregson et al., Carbohydrate Polymers, 38, 255-259 (1999)) and in which
R, radius of container: 30 mm.
L, sample thickness: 10 mm.
r, radius of the probe: 12 mm.
δ, depth of penetration: 0.3 mm.
F, applied force: measured variable from curve slope in N/mm.

Sample 1: Cross-linked PCL530 di-IPTS.

Sample 2: Cross-linked PCL530 di-IPTS (83% w/w)/PCL2000 di-IPTS (17% w/w).

Sample 3: Cross-linked PCL530 di-IPTS (50% w/w)/PCL2000 di-IPTS (50% w/w).

Sample 4: Cross-linked TMPE1014 tri-IPTS.

Sample 5: Cross-linked PTMG2000 di-IPTS.

Sample 6: Cross-linked (CL)₄-PEG1000-(CL)₄ di-IPTS.

Sample 7: Cross-linked (CL)₆-PEG1000-(CL)₆ di-IPTS.

Sample 8: Cross-linked PCL530 di-IPTS (90% w/w)/PCL80000 di-IPTS (10% w/w).

| | Apparent Modulus |
|---|---|
| Sample | [MPa] |
| 1 | 10.7 |
| 2 | 6.4 |
| 3 | 3.4 |
| 4 | 7.0 |
| 5 | 7.2 |
| 6 | 8.7 |
| 7 | 7.7 |
| 8 | 11.5 |

### Example 20

### Thermal Analysis of Different IPTS Derivatives Before and After Cross-Linking at 37° C.

Thermal analysis was carried out by Differential Scanning Calorimetry (DSC) (Mettler Toledo 822°). The samples were sealed in 401 μl μl-crucible pans and their weight was kept between 9-12 mg. The materials were subjected to two consecutive runs: first, they were cooled down from 70° C. to −120° C. and then heated up back to 70° C., at 10° C./min heating or cooling rate.

| | Before cross-linking at | | | After cross-linking at | | |
|---|---|---|---|---|---|---|
| | 37° C. | | | 37° C. | | |
| Material | T_{g}[° C.] | T_{c}[° C.] | Tₘ[° C.] | T_{g}[° C.] | T_{c}[° C.] | Tₘ[° C.] |
| PEG400 di-IPTS | −84 | — | — | −49 | — | — |
| PEG1000 di-IPTS | −79 | −26/−14 | 20 | −42 | −21 | 28 |
| PCL530 di-IPTS | −74 | — | — | −74 | — | — |
| PCL2000 di-IPTS | −70 | 10 | 37/44 | −67 | −22/9 | −1/41 |
| PTMG2000 CL di-IPTS | −96 | −42 | 4 | −87 | −54 | −8 |
| (CL)₄-PEG1000- | −71 | −18 | 12/24 | −72 | — | — |
| (CL)₄di-IPTS | | | | | | |
| (CL)₆-PEG1000- | −69 | −18/2 | 10/27 | −74 | −38 | 13 |
| (CL)₆di-IPTS | | | 37 | | | |
| TMPE1014 tri-IPTS | −73 | −38 | −22 | −67 | — | — |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A responsive polymeric system comprising one or more silicon-containing reactive groups, which system undergoes a hydrolysis-condensation reaction primarily at a predetermined body site in the presence of water and at body temperature, whereby as a result of said hydrolysis-condensation reaction the molecular weight of said polymeric system increases due to polymerization and/or crosslinking, and the rheological and mechanical properties of said polymeric system are changed.

2. The responsive polymeric system of claim 1, wherein said responsive polymeric system is deployable at a predetermined body site via a non-invasive or a minimally invasive surgical procedure.

3. The responsive polymeric system of claim 1, wherein said responsive polymeric system comprises one or more alkoxysilane groups which undergo a hydrolysis-condensation reaction in the presence of water which reaction is effected primarily at a predetermined body site, said reaction resulting in an increase in the molecular weight of the polymeric system and producing a change in the rheological and mechanical properties of said system.

4. The responsive polymeric system of claim 1, wherein said responsive polymeric system comprises one or more silanol groups which undergo a hydrolysis-condensation reaction in presence of water at an appropriate pH, which reaction is effected primarily at a predetermined body site, said reaction resulting in an increase in the molecular weight of the polymeric system and producing a change in the rheological and mechanical properties of said system.

5. The responsive polymeric system of claim 1, wherein said responsive polymeric system is biodegradable whereby the system disappears from the site after a predetermined time.

6. The responsive polymeric system of claim 1, wherein said responsive polymeric system is selectively biodegradable whereby the system reverts to an essentially un-polymerized or non-crosslinked state after a predetermined time.

7. The responsive polymeric system of claim 1, wherein said responsive polymeric system comprises at least one silicon-containing reactive group, said at least one group being a mono, di or tri-functional group.

8. The responsive polymeric system of claim 1, wherein said responsive polymeric system generates a polymer selected from the group consisting of a linear polymer, a block polymer, a graft polymer, a comb polymer, a star-like polymer, a crosslinked polymer and combinations thereof.

9. The responsive polymeric system of claim 1, wherein said responsive polymeric system also comprises additional reactive groups selected from the group consisting of hydroxyl, carboxyl, thiol, amine, isocyanate, thioisocyanate and double bond-containing active groups, and combinations thereof.

10. The responsive polymeric system of claim 1, wherein said increase in the molecular weight of the polymeric system and said change in its rheological and mechanical properties is partial, and the system is still able to retain some degree of flowability

11. The responsive polymeric system of claim 1, wherein said responsive polymeric system comprises more than one component that form covalent bonds between them or generate physical blends or interpenetrating or pseudo-interpenetrating networks and combinations thereof, at the predetermined body site.

12. The responsive polymeric system of claim 1, wherein said responsive polymeric system contains at least one biomolecule to be delivered into the body.

13. The responsive polymeric system of claim 1, wherein said responsive polymeric system contains living cells or a material of tissue origin.

14. The responsive polymeric system of claim 1, wherein said responsive polymeric system also comprises a solid component, wherein said solid component is a macro, micro or nano-sized material selected from the group consisting of a polymer, a ceramic material, a metal, a carbon, a biological material, and combinations thereof, wherein said solid component is selected from the group consisting of a particle, a sphere, a capsule, a rod, a slab, a fiber, a mesh, a ribbon, a web, a non-woven structure, a fabric, an amorphous lattice structure, a filament wound structure, a honeycomb structure or a braided structure, and combinations thereof, and wherein said solid component may be hollow or porous, and combinations thereof.

15. The responsive polymeric system of claim 14, wherein said solid component possesses reactive moieties capable of reacting with the reactive groups present in the responsive polymeric system.

16. The responsive polymeric system of claim 14, wherein said solid component is a biodegradable material.

17. The responsive polymeric system of claim 14, wherein said solid component is a ceramic material selected from the group consisting of tricalcium phosphate, hydroxyapatite, and combinations thereof.

18. The responsive polymeric system of claim 14, wherein said solid component is of tissue source.

19. The responsive polymeric system of claim 14, wherein said solid component comprises a material selected from the group consisting of elastin, a collagenous material, albumin, a fibrinous material, demineralized tissue, an acellular tissue matrix, and combinations thereof.

20. The responsive polymeric system of claim 14, wherein said solid component contains at least one biomolecule, to be delivered into the body.

21. The responsive polymeric system of claim 14, wherein said solid component contains living cells.

22. The responsive polymeric system of claim 14, wherein said solid component is chemically or physically bound to said responsive polymeric system of claim 1.

23. The responsive polymeric system of claim 1, wherein said responsive polymeric system is a low molecular weight polymer capable of being deployed at a predetermined body site by minimally invasive procedures, said low molecular weight polymer being selected from the group consisting of polyoxyalkylene, polyester, polyurethane, polyamide, polycarbonate, polyanhydride, polyorthoesters, polyurea, polypeptide, polyalkylene, acrylic or methacrylic polymers, polysaccharide, and combinations thereof.

24. The responsive polymeric system of claim 23, wherein said responsive polymeric system is biodegradable or selectively biodegradable.

25. The responsive polymeric system of claim 1, wherein said polymeric responsive system is also capable of undergoing a transition that results in a sharp increase in viscosity in response to a predetermined trigger at a predetermined body site, wherein said transition results in an increase in the viscosity of said responsive polymeric system by at least about 2 times.

26. The responsive polymeric system of claim 25, wherein said predetermined trigger is temperature, wherein said increase in viscosity takes place as a result of heating from a lower temperature to body temperature.

27. The responsive polymeric system of claim 26, wherein said polymeric responsive system comprises water or an aqueous-based solvent.

28. The responsive polymeric system of claim 26, wherein said responsive polymeric component is biodegradable.

29. The responsive polymeric system of claim 26, wherein said responsive polymeric system is selected from the group consisting of a polyoxyalkylene polymer, a block copolymer comprising polyethylene oxide (PEO) and polypropylene oxide (PPO) selected from a group consisting of a diblock, a triblock or a multiblock, a segmented block copolymer comprising polyethylene oxide (PEO) and polypropylene oxide (PPO) chains, wherein said PEO and PPO chains are connected via a chain extender, a poly(alkyl-co-oxyalkylene) copolymer having the formula R—(OCH₂CH)ₙ—OH, where R is an hydrophobic monofunctional segment selected from a group consisting of poly(tetramethylene glycol), poly(caprolactone), poly(lactic acid), poly(siloxane) and combinations thereof, a poly(alkyl-co-oxyalkylene) copolymer having the formula [−R′—(OCH₂CH)ₙ—O]ₚH, where R′ is a bifunctional or multifunctional hydrophobic segment, a poly(N-alkyl substituted acrylamide), preferably poly(N-isopropyl acrylamide), cellulose and cellulose derivatives, and combinations thereof.

30. The responsive polymeric system of claim 26, wherein said responsive component is a segmented block copolymer comprising polyethylene oxide (PEO) and polypropylene oxide (PPO) chains, wherein said PEO and PPO chains are connected via a chain extender, wherein said chain extender comprises a component selected from the group consisting of phosgene, aliphatic or aromatic dicarboxylic acids or their acyl chlorides or anhydrides, cyanuric chloride, dicyclohexylcarbodiimide (DCC), hexamethylene diisocyanate (HDI), methylene bisphenyldiisocyanate (MDI), and other aliphatic or aromatic diisocyanates.

31. The responsive polymeric system of claim 29, wherein said poly(N-alkyl substituted acrylamide) is a copolymer comprising alkoxysilane-containing vinyl monomers.

32. The responsive polymeric system of claim 1, wherein said responsive polymeric system further comprises other polymers that are responsive to other stimuli selected from the group consisting of temperature, pH, ionic strength, electric and magnetic fields, energy sources covering a broad range of wavelengths selected from the group consisting of ultraviolet, visible, infrared, microwave, ultrasound, electron beam and x-rays radiation, fluids and biological species, and combinations thereof.

33. The responsive polymeric system of claim 32, wherein said additional component is capable of undergoing a transition as a result of an increase in temperature that results in a sharp increase in viscosity of at least about 2 times.

34. The responsive polymeric system of claim 1, wherein said responsive component contains biologically or pharmacologically active molecule/s, to be delivered into the body following a unimodal or multimodal time dependent release kinetics, as the molecular weight of the polymeric system as well as its rheological and mechanical properties change at the predetermined body site.

35. The responsive polymeric system of claim 1, whenever used as a sealant, a coating and lubricant, a transient barrier for the prevention of post-surgical adhesions, a matrix for the unimodal or multimodal controlled release of biologically active agents, and in the area of Tissue Engineering and the field of Gene Therapy.

36. The responsive polymeric system of claim 1, wherein said responsive polymeric system contains biologically or pharmacologically active molecule/s, wherein said active molecules are covalently bound to said polymeric system.

37. The responsive polymeric system of claim 1, wherein said responsive polymeric system contains biologically or pharmacologically active molecule/s, wherein said active molecules are covalently bound to the polymeric system via silicon-containing reactive groups present in said responsive polymeric system.

38. The responsive polymeric system of claim 1, wherein said silicon moieties serve as nuclei for the deposition or crystallization of various materials.

39. The responsive polymeric system of claim 1, wherein said silicon moieties serve as nuclei for the deposition or crystallization of hydroxyapatite or other calcium phosphate derivatives for bone regeneration induction at a predetermined body site.

40. The responsive polymeric system of claim 1, wherein said responsive polymeric system is a water solution or a gel comprising a molecule containing silicon-containing reactive groups and a natural or synthetic macromolecule and combinations thereof containing functional groups capable of reacting with said silicon-containing reactive groups at a predetermined body site.

41. The responsive polymeric system of claim 1, wherein said responsive polymeric system is a water solution or a gel comprising a molecule containing silicon-containing reactive groups and functional groups capable of reacting with said silicon-containing reactive groups at a predetermined body site.

42. The responsive polymeric system of claim 40, wherein said macromolecule comprises polymer or oligomer selected from the group consisting of alginates and its derivatives, hyaluronic acid and its derivatives, collagen, gelatin, chitosan and its derivatives, agarose, cellulose and its drivatives, oligoHEMA, polyacrylic acid, polyvinyl alcohol, polyethylene oxide, TMPO, peptides, proteins, and combinations thereof.
